(19) United States
(12) Patent Application Publication
(10) Pub. No.: US 2011/0136109 A1
(43) Pub. Date: Jun. 9, 2011

(54) System And Method For Cycling Liquid Samples Through A Series Of Temperature Excursions

(75) Inventors: Andreas Drechsler, Baar (CH); Emad Sarofim, Hagendorn (CH); Claudio Cherubini, Cham (CH); Stephan Korner, Cham (CH)

(73) Assignee: ROCHE DIAGNOSTICS OPERATIONS, INC., Indianapolis, IN (US)

(21) Appl. No.: 12/963,169

(22) Filed: Dec. 8, 2010

(30) Foreign Application Priority Data
Dec. 9, 2009 (EP) 09178575.8

(51) Int. Cl. C12Q 1/68; C12M 1/34; C12M 1/00

(52) U.S. Cl. 435/6.1; 435/287.2; 435/283.1

(57) ABSTRACT

A system and method for cycling liquid samples through a series of temperature excursions are disclosed. Provided are open-top reaction vessels for containing the samples, which may be enclosed by one or more covers. A temperature-controlled block for generating or adsorbing heat is coupled thermally to the reaction vessels. A detection arrangement is disposed in an emission beam path to detect radiation emitted from the samples through the covers. A heating arrangement for generating heat includes a heating element that is both disposed between the reaction vessels and the detection arrangement and coupled thermally to the covers. The heating element includes an optically transparent substrate provided with one or more opaque heating lines, the heating lines being disposed in the emission beam path in a manner to obtain a predetermined minimum optical transmission of the heating element. A controller, set up to control cycling of the samples, is provided.

### TECHNICAL FIELD

The present disclosure relates generally to clinical analysis and medical diagnostics and more particularly to an automated system and method for cycling liquid samples through a series of temperature excursions.

### BACKGROUND

Nucleic acids (DNA=deoxyribonucleic acid, RNA=ri-bonucleic acid) are frequently used as a starting material for various analyses and assays in medical and pharmaceutical research, clinical diagnosis and genetic fingerprinting which typically require high quantity nucleic acids input.

As a matter of routine, major quantities of nucleic acids can readily be obtained by means of in-vitro amplification techniques, e.g., using the well-known polymerase chain reaction (PCR). The amplification of nucleic acids based on PCR has been extensively described in patent literature, for instance, in U.S. Pat. Nos. 4,683,303, 4,683,195, 4,800,159 and 4,965,188. Basically, in PCR, the samples are repeatedly put through a sequence of amplification steps (“cycled”) which includes melting the nucleic acids to obtain denaturated single polynucleotide strands, annealing short primers to the strands, and extending those primers to synthesize new polynucleotide strands along the denaturated strands to make new copies of double-stranded nucleic acids. The amplification of nucleic acids requires the samples to be cycled through a series of temperature excursions in which predetermined temperatures are kept constant for specific time intervals. Stated more particularly, the temperature of the samples usually is raised to around 90° C. for denaturing the nucleic acids and lowered to 40° C. to 70° C. for annealing and primer extension along the polynucleotide strands.

In daily routine, commercially available apparatus (“thermal cyclers”) are used for cycling reaction mixtures through the temperature excursions employing a temperature-controlled (thermal) block for heating and/or cooling the samples. As for instance is described in U.S. patent application 2005/0145273 A1, temperature-control of the thermal block can, e.g., be based on thermoelectric heating and cooling devices utilizing the Peltier (or thermoelectric) effect. Connected to a DC power source, each of the Peltier devices functions as a heat pump which can produce or adsorb heat to thereby heat or cool the samples depending upon the direction of the electric current applied. Accordingly, the temperature of the samples can be changed according to predefined cycling protocols as specified by the user by applying varying electric currents to the Peltier devices. Due to the fact that reaction rates in the PCR reactions strongly vary with temperature, it is desirable that the samples have temperatures throughout the thermo-cycling process that are as uniform as reasonably possible since even small variations can cause a failure or undesirable outcome of the amplification process. Therefore, temperature errors and variations between the samples should be minimized.

In PCR, open-top reaction vessels typically are enclosed by covers such as sealing foils or lids in order to avoid evaporation of the reaction mixtures contained and to shield them from external influences. It is convenient to use transparent covers which allow for an optical detection of the reaction products contained in the reaction vessels even during progress of the reaction.

Usually, the reaction vessels are not completely filled with reaction mixtures, each of which thereby having an air or other gas gap in-between the reaction mixture and the underside of the cover. Hence, when thermally cycling the reaction mixtures, formation of condensation within each of the reaction vessels in particular on the undersides of the covers is likely to occur. However, such condensation reduces the optical transmission of the covers and thus interferes with the optical detection of the reaction products. Otherwise, condensation results in variations of the reaction mixtures and can cause an undesirable outcome or even failure of the amplification process. Therefore, condensation on the inner walls and, in particular, on the undersides of the transparent covers of the reaction vessels should be minimized.

In the prior art this problem has been addressed by several technical solutions. According to one prior art solution, a transparent cover is being provided with a layer of indium-tin-oxide (ITO) which produces Ohmic heat when an electrical current flows through it. The production of such cover layers, however, is expensive and due to thermal and mechanical stress, the cover layer may separate from the substrate (transparent cover) which compromises the optical and thermal properties of the arrangement. Otherwise, providing for a non-uniform distribution of heating power is very difficult to realize, and, layer thickness and electric resistance is limited in view of the desired layer transparency.

According to another prior art solution, heating of the transparent cover is performed only outside the optical path, but immediately adjacent the transparent cover portions. Therefore, condensation is prevented due to heating of the transparent cover in these areas by heat flow from the adjacent areas. This spatial requirement is a major drawback of this solution. Another drawback is a quite cumbersome positioning and strict requirements imposed on tolerances especially when the number of vessels grows and hence the dimension of cover portions which are transmitted by radiation decreases.

### SUMMARY

An improved system and method for cycling liquid samples through a series of temperature excursions, which allow for an improved optical online detection, are described.

In one embodiment, a system for cycling liquid samples through a series of temperature excursions is disclosed. The system may comprise a plurality of open-top reaction vessels for containing the samples, the reaction vessels being enclosed by one or more covers; a temperature-controlled thermal block for generating or adsorbing heat thermally coupled to the reaction vessels; and a detection arrangement for detecting radiation disposed in an emission beam path to detect emission beams emitted from the samples received through the one or more covers. The system may comprises also a heating arrangement for generating heat including a heating element disposed between the reaction vessels and the detection arrangement and being thermally coupled to the one or more covers, the heating element including an optically transparent substrate provided with one or more opaque heating lines, the heating lines being disposed in the emission beam path in a manner to obtain a predetermined minimum optical transmission of the heating element; and a controller, set up to control cycling of the samples.

In another embodiment, a heating arrangement for heating one or more covers enclosing a plurality of reaction vessels for containing liquid samples is disclosed. The heating arrangement may include a heating element disposed between the one or more covers and a detection arrangement disposed along an emission beam path for detecting emission beams emitted from the samples received through the one or more covers. The heating element may include an optically transparent substrate provided with one or more opaque heating lines disposed in the emission beam path in a manner to obtain a predetermined minimum optical transmission of the heating arrangement.

In still another embodiment, a method for cycling liquid samples through a series of temperature excursions is disclosed. The method may comprise providing the liquid samples in a plurality of open-top reaction vessels enclosed by one or more covers; thermally cycling the samples; and detecting emission beams emitted from the samples and received through the one or more covers along an emission beam path. The method may comprise also heating the one or more covers by a heating arrangement including a heating element having a transparent substrate provided with one or more opaque heating lines disposed in the emission beam path in a manner to obtain a predetermined minimum optical transmission of the heating element.

Other and further features and advantages of the various embodiments of the invention will appear more fully from the accompanying drawings and the following detailed description.

### BRIEF DESCRIPTION OF THE DRAWINGS

In the drawings, like elements are indicated with like numbers, and in which:

FIG. 1 is a schematic diagram illustrating an exemplary embodiment of the system of the invention;

FIG. 2 is a schematic diagram depicting an exemplary embodiment of the heating arrangement of the system of FIG. 1;

FIG. 3 is a schematic illustration depicting an exemplary non-uniform area density of heating power of the heating arrangement of the system of FIG. 1;

FIG. 4 is a schematic diagram illustrating varying electric resistances of the heating lines of the heating arrangement of FIG. 3;

FIGS. 5A-5B are schematic diagrams depicting a varying line width of an individual heating line of a variant of the heating arrangement of the system of FIG. 1;

FIGS. 6A-6B are schematic diagrams depicting a varying line height of an individual heating line of another variant of the heating arrangement of the system of FIG. 1;

FIGS. 7A-7B are schematic diagrams depicting a varying line material of an individual heating line of another variant of the heating arrangement of the system of FIG. 1;

FIGS. 8A-8B are schematic diagrams depicting a varying area density of plural portions of a meandering heating line of the heating arrangement of the system of FIG. 1;

FIGS. 9A-9C are schematic diagrams illustrating different exemplary embodiments of the heating arrangement of FIG. 1;

FIGS. 10A-10B are schematic diagrams illustrating another embodiment of the heating arrangement of FIG. 1; and

FIGS. 11A-11B are schematic diagrams further illustrating the heating arrangement of FIGS. 10A-10B.

### DETAILED DESCRIPTION

The embodiments described hereafter are set forth to aid the understanding of the invention, but are not to be construed as limiting.

In some embodiments, according to a first aspect of the invention, a system for the automated cycling of liquid samples through a series of temperature excursions is disclosed. The system can be configured in various ways in accordance with specific demands of the user. Liquid samples can be reaction mixtures containing biological material in which nucleic acids can potentially be found. The system can be configured to cycle liquid samples in a manner to accomplish a polymerase chain reaction, a reverse transcription-polymerase chain reaction or any other chemical reaction of the nucleic acid amplification type. Samples for cycling by the system of the invention, however, are not limited to biological reaction mixtures but may also include any other fluid of interest for which it is desired to perform thermal cycling such as, but not limited to, cells, tissues, micro-organisms or non-biological fluids. Samples can be mixed with one or more reagents, e.g., with a view of amplifying nucleic acids contained therein in order to obtain reaction products which can be optically detected. As used herein, the term “reagent” is used to indicate any liquid which can be mixed with the samples and/or one or more other reagents. In the more strict sense of the term, reagents include components which can react with the sample. Reagents, however, can also be non-reacting fluids such as buffers and diluting fluids.

In another embodiment, the system may comprise a plurality of open-top reaction vessels for containing the liquid samples. An opening of each of the reaction vessels is provided with a cover such as a sealing foil or a lid for enclosing the reaction vessel.

In some embodiments, a planar multi-well plate having a two-dimensional array of cavities or wells is used for providing the reaction vessels. In some embodiments, the wells are covered by one cover. In some embodiments, each well is covered by an individual cover. In some embodiments, the plate consists of plastic material intended for single use only. In some embodiments, the reaction vessels can be manually or automatically filled with the samples which may occupy at least some or all of the reaction vessels. In some embodiments, the covers can be punctured to fill the reaction vessels with the samples and optionally one or more other fluids. In some embodiments, the one or more covers can rest on, attach to or seal tightly with the reaction vessels.

The system may further include a temperature-controlled (thermal) block for generating or absorbing heat which is thermally coupled to the reaction vessels to heat and/or cool the reaction vessels in order to thermally cycle the liquid samples contained therein. In some embodiments, the thermal block is provided with a plurality of wells shaped to receive the reaction vessels, e.g., provided by a multi-well plate. In some embodiments, the thermal block includes one or more thermoelectric heating and cooling devices utilizing the Peltier effect, each of which functioning as a heat pump to produce or adsorb heat for heating and/or cooling the reaction vessels depending upon the direction of the electric current applied.

The system may further include a detection arrangement for optically detecting radiation which is disposed along an emission beam path (optical path) and positioned to detect emission beams emitted from the reaction vessels received through the one or more covers. In some embodiments, the detection arrangement includes one or more detectors for optically detecting the emitted light such as, but not limited to, charge coupled devices (CCDs), diode arrays, photomultiplier tube arrays, charge injection devices (CIDs), CMOS detectors and avalanche photo diodes.

In some embodiments, the detection arrangement also includes one or more excitation light sources to excite emission of the emission beams from the samples. In some embodiments, the detection arrangement further includes light guiding elements such as, but not limited to, lenses and mirrors and/or light separating elements such as, but not limited to, transmission gratings, reflective gratings and prisms.

Basically, the one or more covers are optically transparent or at least include optically transparent portions which allow radiation such as excitation light to be transmitted to the samples and emitted (e.g. fluorescent) light to be transmitted back to the one or more detectors, e.g., during thermal cycling of the samples.

The system may further include a heating arrangement for heating the one or more covers including a heating element thermally coupled to the covers of the reaction vessels. The heating element includes an optically transparent substrate such as, but not limited to, a plate-like substrate provided with one or more opaque (i.e. non-transparent) heating lines to heat the substrate by generating Ohmic heat. In the heating element, the one or more heating lines are disposed in the emission beam path in a manner to obtain a predetermined minimum optical transmission of the heating element. The heating lines may be, e.g., embedded in the substrate and/or secured to a surface thereof. The heating lines can be formed by conventional thin film technology based on depositing a film of conductive material on a surface of the substrate, e.g., by use of chemical vapor deposition (CVD), physical vapor deposition (PVD) or sputtering, followed by patterning the film, e.g., by use of a mask. The production of the heating lines can be based on conventional lithographic technology.

In some embodiments, the substrate is made of glass such as, but not limited to, borosilicate glass. In some embodiments, the heating lines are made of metallic material such as, but not limited to, platinum or platinum alloy.

In some embodiments, the substrate includes one or more sensors for sensing temperatures of the substrate.

In some embodiments, the heating element has a minimum optical transmission of 50%, particularly of 70%, and more particularly of 85% with respect to light emitted from the samples.

In some embodiments, a covered portion of an irradiated opening area at the opening of individual reaction vessels covered by the one or more opaque heating lines is less than 20%, in particular less than 10%. As used herein, the term “irradiated opening area” denotes a portion of a cross-sectional (i.e. geometric) opening area of the opening of each of the reaction vessels irradiated by radiation emerging from the sample contained therein. Specifically, in case of irradiating the whole cross-sectional opening area of an individual reaction vessel, the irradiated opening area is identical to the cross-sectional opening area of the reaction vessel concerned. Otherwise, the irradiated opening area can also be smaller than the cross-sectional opening area of the reaction vessel concerned. Furthermore, the term “covered portion” denotes a portion of the irradiated opening area of an individual reaction vessel shadowed by the opaque heating lines. Accordingly, radiation emitted from the sample contained in individual reaction vessels cannot penetrate the covered portion of the irradiated opening area. Hence, the irradiated opening area of individual reaction vessels is composed of the covered portion shadowing radiation emerging from the sample contained therein and a non-covered portion enabling transmission of the radiation.

In some embodiments, individual heating lines have a width of less than 150 μm, preferably less than 120 μm, and more preferably are in a range of from 10 μm to a few 10 μm such as, but not limited to, 70 μm.

In some embodiments, adjacent heating lines and/or adjacent portions of individual heating lines have an inter-distance of more than 100 μm, and preferably are in a range of from 100 μm, to a few millimeters. Specifically, a covered portion with respect to an irradiated opening area of individual reaction vessels of less than 20%, in particular less than 10%, can be obtained.

As used herein, the term “width” denotes a linear dimension of the heating lines as measured orthogonal to the extension of the heating lines in a plane of the substrate. The term “inter-distance” denotes a linear dimension in-between adjacent heating lines and/or adjacent portions of individual heating lines as measured orthogonal to the extension of the heating lines and portions thereof, respectively, in a plane of the substrate. Otherwise, the term “height” denotes a linear dimension of the heating lines as measured orthogonal to the extension of the heating lines and orthogonal to the plane of the substrate.

The system may further include a controller set up to control thermal cycling of the samples. In some embodiments, the controller is embodied as programmable logic controller running a machine-readable program provided with instructions to perform operations in accordance with a predetermined process operation plan for thermally cycling the samples. The controller is electrically connected to the system components which require control which include the thermal block and, if present, the one or more temperature sensors of the substrate.

Contrary to the prior art solutions as above-detailed which aim at keeping heating lines out of the detection path, the present invention proposes a solution where, in the embodiments of the invention, the heating lines can be disposed in areas (irradiated opening areas) irradiated by detection radiation. According to the embodiments of the present invention, it has been found that the presence of the heating lines in the optical path can be tolerated if the diameter of the heating lines is small enough and if on the other hand their density is high enough to provide sufficient heating power without overcharging the individual heating lines. This setup—comparably small diameter of the heating lines of sufficient density and narrow spacing—not only provides sufficient transparency in the detection path but also ensures that even if positioning and/or production tolerances are present the transparency of different optical detection pathways remains substantially constant. Hence, the embodiments of the system advantageously allows for an optical detection of light received through the one or more covers even in case of locating the heating lines of the heating element in the optical path of the emission beams. Thus, the heating element can be disposed between the reaction vessels and the detection arrangement without a need to exactly position the heating element with respect to the reaction vessels in order to avoid the heating lines being located within the optical path of the emitted light which remarkably facilitates the design (set-up) of the system without a need to keep small tolerances. A reduction of intensities of the emission beams due to major shadowing and/or scattering effects caused by the heating lines can advantageously be avoided due to many comparably small heating lines instead of only a few comparably thick heating lines. The optical transmission of the heating arrangement is as high as reasonably possible to permit the user to optically detect the emitted light in a reliable and satisfactory manner. Otherwise, due to the many small heating lines, compared to the case of having only few thicker heating lines, variations of the heating lines covered irradiated opening areas of the reaction vessels can advantageously be reduced.

Specifically, the heating arrangement of the system can readily be used for various multi-well plates having array sizes which are different with respect to each other. It especially permits the user to visually or optically detect the contents of the reaction vessels, e.g., during the course of the reaction and thereby achieve real-time detection of the progress of the reaction.

The heating arrangement can be made compact in design to yield high stability and less susceptibility to faults. The heating arrangement further allows the reaction vessels to be enclosed with covers to prevent evaporation of the reaction mixtures and without experiencing condensation by heating the covers. Therefore, undesirable condensation on the covers which can reduce optical transmission thereof is advantageously reduced or even avoided. Due to the heating arrangement, the reaction vessels can also be more homogenously heated to avoid temperature variations so as to enable that chemical reactions in the reaction vessels take place in a similar manner.

In various embodiments of the system, in particular in case of providing the reaction vessels by a multi-well plate, edge effects may cause temperature differences between outer and inner reaction vessels. Stated more particularly, due to their greater exposure to the atmosphere and/or to other system components, outer reaction vessels typically have a lower temperature than inner reaction vessels. In order to circumvent such drawback, in some embodiments of the system, the one or more heating lines are being operable to yield a non-uniform area density of heating power (i.e. heating power per unit area) with respect to an area of the substrate thermally coupled to the one or more covers. The non-uniform area density of heating power can compensate for such edge effects so as to obtain a uniform (homogenous) temperature of the reaction vessels.

In some embodiments of the system, individual heating lines are designed to have a varying electric resistance over their extensions to thereby obtain a non-uniform area density of heating power.

In some embodiments, individual heating lines vary in one or more of the following line characteristics selected from the group consisting of line width, line height and line material to yield a varying electric resistance over their extensions to thereby obtain a non-uniform area density of heating power.

In some embodiments, an area density of the heating lines and/or portions of individual heating lines with respect to an area of the substrate coupled to the one or more covers can vary to thereby obtain a non-uniform area density of heating power.

In some embodiments, the heating arrangement includes plural heating lines having various inter-distances of neighboring heating lines to thereby obtain a non-uniform area density of heating power.

In some embodiments, the heating arrangement includes individual heating lines having various inter-distances of neighboring heating line portions to thereby obtain a non-uniform area density of heating power.

In some embodiments, the heating arrangement includes one or more meandering heating lines, each of which including plural neighboring portions having various inter-distances to thereby obtain a non-uniform area density of heating power.

In some embodiments, in the heating arrangement the one or more heating lines are operable to yield different area densities of heating power in different regions of the substrate. Specifically, the one or more heating lines can be operable to yield a first area density of heating power in a first region of the substrate being lower than a second area density of heating power in at least one second region of the substrate. In some embodiments, the first region is a central region of the substrate while the second region is an edge region of the substrate surrounding the central region. In some embodiments, a ratio of the first area density of heating power to the second area density of heating power is in a range of from about 1 to about 1.5 through about 1 to about 10, and in other embodiments in a range of from about 1 to about 2 through about 1 to about 3, to thereby obtain a homogenous temperature of the reaction vessels.

In some embodiments, the heating arrangement includes (only) one heating circuit consisting of a resistor network connected to one electric power source to obtain a non-uniform area density of heating power with respect to a unit area of the substrate. As used herein, the term “resistor network” denotes an electrically connected network of resistors which can be similar or different with respect to each other. In order to enable a non-uniform area density of heating power, at least two resistors are different with respect to each other.

In some embodiments, the heating arrangement includes at least two separate heating circuits, each of which having separate electric connectors which are selectively connectable to one or more electric power sources. This feature enables the additional function of selectively operating the heating line circuits by applying different currents and/or voltages to thereby obtain a non-uniform area density of heating power with respect to a unit area of the substrate.

In some embodiments, in the heating arrangement, the substrate is adapted to force the reaction vessels onto the thermal block. This feature advantageously serves two additional functions. The first is to improve the sealing effect of the covers of the reaction vessels thereby helping avoid evaporation of the reaction mixtures and shielding the samples from external influences. The second is to provide for a good thermal contact to make the heat distribution uniform.

In other embodiments, according to a second aspect of the invention, a new heating arrangement for heating one or more covers enclosing a plurality of liquid vessels for containing liquid samples is disclosed. The heating arrangement includes a heating element disposed between the one or more covers and a detection arrangement disposed along an emission beam path for detecting light emitted from the samples and received through the one or more covers. The heating element includes an optically transparent substrate provided with one or more heating lines disposed in the optical path in a manner to obtain a predetermined minimum optical transmission of the heating element. The heating arrangement of the second aspect of the invention can be used in a system for cycling liquid samples through a series of temperature excursions which can be similar to the above-described system of the invention.

In still other embodiments, according to a third aspect of the invention, a new method for cycling liquid samples through a series of temperature excursions is disclosed. In one embodiment, the method includes an act of providing the liquid samples in a plurality of open-top reaction vessels enclosed by one or more covers. The method may include further act of thermally cycling the samples. The method includes a yet further act of detecting light emitted from the samples and received through the one or more covers along an emission beam path. The method includes a yet further act of heating the one or more covers by a heating arrangement including a heating element comprising a transparent substrate provided with one or more heating lines disposed in the emission beam path in a manner to obtain a predetermined minimum optical transmission of the heating element.

In some embodiments, the method is implemented by the above-described system of the invention. Hence, in some embodiments, the method includes an act of providing a system as-above described which may be embodied according to any one or any combination of the above-described embodiments.

In some embodiments, the method includes a further act of operating the one or more heating lines to yield a non-uniform area density of heating power with respect to an area of the substrate thermally coupled to the one or more covers so as to obtain a uniform temperature of the reaction vessels.

In some embodiments, the method includes an act of operating the one or more heating lines to yield different area densities of heating power in different regions of the substrate. Specifically, the method can include an act of operating the one or more heating lines to yield a first area density of heating power in a central region of the substrate being lower than a second area density of heating power in an edge region of the substrate surrounding the central region. Using a system according to any of the embodiments as above-described in connection with the first aspect of the invention, the controller is set up to control the method of the invention.

The above-described embodiments of the various aspects of the invention may be used alone or in any combination thereof without departing from the scope of the invention.

By way of illustration, specific exemplary embodiments in which the invention may be practiced are described hereafter with reference made to the figures.

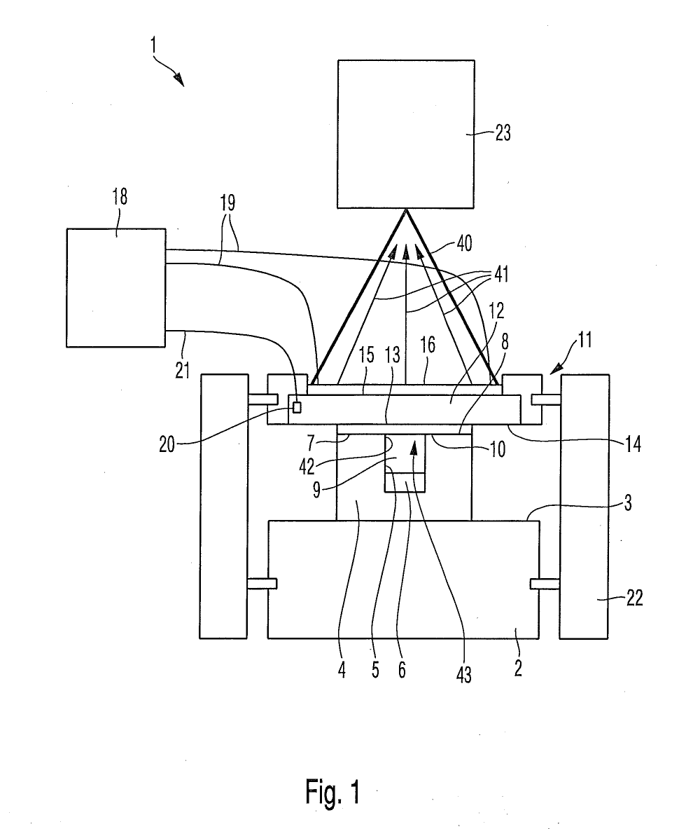

With reference to FIG. 1, by means of a schematic diagram, an exemplary embodiment of the system 1 for the automated cycling of liquid samples is explained. The system 1 may be used to cycle samples including biological material, e.g., to accomplish a polymerase chain reaction of nucleic acids contained therein. The samples are mixed with one or more reagents with a view of amplifying the nucleic acids which can be optically detected.

Accordingly, the system 1 for thermocycling liquid samples includes a temperature-controlled thermal block 2 which, e.g., includes a plurality of thermoelectric heating and cooling devices utilizing the Peltier effect. Each of the Peltier devices functions as a heat pump to produce or adsorb heat depending upon the direction of the electric current applied (not further detailed). The thermal block 2 can be heated according to predefined temperature profiles so as to change and hold various temperatures for a predetermined amount of time. Those of skill in the art will appreciate that the Peltier devices can be replaced by any other type of heaters such as resistive heaters.

An upper face 3 of the thermal block 2 supports a planar multi-well plate 4 which comprises a main body provided with a two-dimensional array of cavities or wells 5 (i.e., open-top reaction vessels). Although only one well 5 is shown in FIG. 1 for the purpose of illustration, the rectangular array may, e.g., include 8×12 wells (96 wells total), 6×10 wells (60 total), 16×24 wells (384 total), 32×48 wells (1536 total), or any other number and arrangement that would be compatible with the automated system 1 for thermocycling of liquid samples. The footprint of the multi-well plate 4 may be, e.g., about 127 mm in length and about 85 mm in width, while those of skill in the art will recognize that the multi-well plate 4 can be formed in dimensions other than those specified herein. The multi-well plate 4 may, e.g., consist of plastic material such as, but not limited to, polypropylene, polystyrene and polyethylene. It may be, e.g., intended for single use only so that it is filled with liquid samples and/or reagent mixtures 6 for a single experiment and is thereafter discarded. Alternatively, the multi-well plate 4 may be intended for multiple-use, wherein it is operable for use in a plurality of experiments or sets of experiments.

Accordingly, heat can be transferred between the thermal block 2 and the multi-well plate 4 to vary the temperature of liquid samples 6 contained in the wells 5 to be processed. The thermal block 2 may be, e.g., provided with a plurality of cavities (not illustrated) shaped to receive the wells 5 of the multi-well plate 4. Stated more particularly, the outer contours of the wells 5 are conform in shape to the inner profiles of the cavities of the thermal block 2 such that the multi-well plate 4 can be placed over the thermal block 2 with the wells 5 thereof resting inside the cavities of the thermal block 2 in a close fit with full contact for thermal communication between the thermal block 2 and the wells 5. The contours of the wells 5 may be, e.g., conical to achieve efficient heat transfer. Alternatively, the multi-well plate 4 can, e.g., be replaced by individual reaction vessels put into the cavities of the thermal block 2. Hence, by use of the thermal block, the samples and/or reagent mixtures 6 contained in the wells 5 of the multi-well plate 4 can be cycled through pre-defined temperature profiles.

An optically transparent cover or sealing foil 7 encloses (i.e. tightly seals) openings 42 of the wells 5 in order to prevent evaporation of the liquid samples 6 contained therein and to shield the samples 6 from external influences. The sealing foil 7 is fixedly secured to a circular or square-shaped rim portion 8 of the wells 5 surrounding the openings 42. The sealing foil 7 may, e.g., comprise a durable, generally optically transparent material, such as an optically clear film exhibiting low fluorescence when exposed to excitation light. The sealing foil 7 may, e.g., comprise glass, quartz, polystyrene and polyethylene. It may, e.g., also comprise one or more compliant coatings and/or one or more adhesives such as a pressure sensitive adhesive or hot melt adhesive to be secured to the rim (edge) portions 8 of the wells 5.

As illustrated in FIG. 1, the wells 5 usually are not completely filled with reaction mixtures 6 thereby having an air gap 9 in-between the reaction mixture 6 and a lower face 10 of the sealing foil 7.

The system 1 further includes a heating arrangement 11 thermally coupled to the sealing foil 7 enclosing the wells 5. The heating arrangement 11 includes a resistive heating element 16 provided with an optically transparent plate-like substrate 12 placed above the sealing foil 7. Stated more particularly, a lower face 14 of the substrate 12 is placed on an upper face 13 of the sealing foil 7 in a close fit with full contact for thermal communication between the substrate 12 and the sealing foil 7.

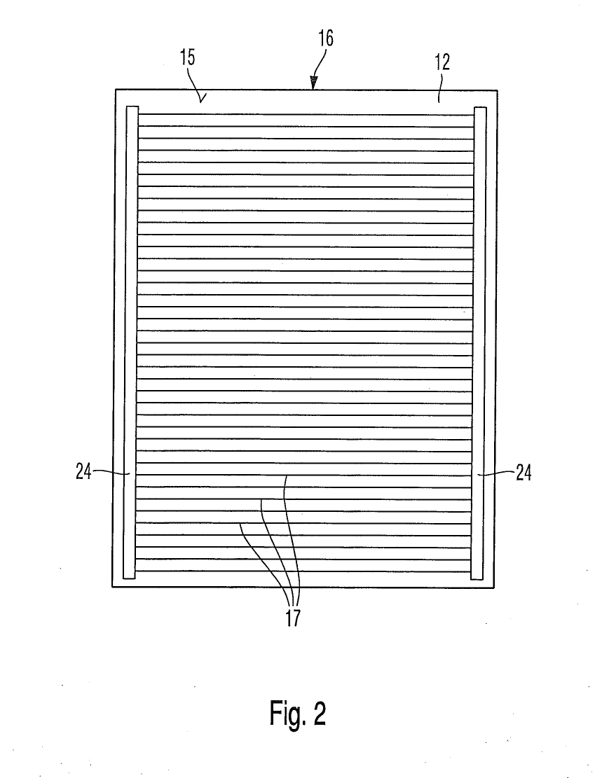

On an upper face 15 thereof, the substrate 12 is provided with a plurality of thin opaque resistive heating lines 17 to heat the substrate 12 by generating Ohmic heat. As illustrated in FIG. 2, the heating lines 17 are positioned in a manner to heat the whole substrate 12. The heating lines 17 may be, e.g., sputter deposited, lithographically deposited, vapor deposited, thin layer coated or can be formed by any other methods. Alternatively, the substrate 12 may include, e.g., internally positioned heating lines embedded within the substrate 12 and be, e.g., positioned during molding of the substrate 12. The heating lines may be, e.g., positioned within channels or ducts formed within the substrate 12. The channels or ducts may be molded into the substrate 12 during its fabrication or subsequently formed by chemical or mechanical methods such as etching or drilling. While not shown in the figures, the resistive heating lines 17 on the upper face 15 can be covered by a protective layer to avoid degeneration of the heating lines 17 and/or to protect them against environmental influences.

With continued reference to FIG. 1, a controller 18 which includes a power source (not illustrated) is operatively coupled to the resistive heating element 16 to output a control signal (voltage) by electric lines 19 to regulate a desired thermal output of the heating element 16. The thermal output is varied in response to an input from sensor 20 placed within the substrate 12 to sense a temperature of the substrate 12. The sensor 20 is electrically connected to the controller 18 by electric line 21. Additionally, the controller 18 is operatively coupled to the thermal block 2 to output a control signal to regulate a desired thermal output of the thermal block 2 (not further detailed in FIG. 1). The thermal output may be, e.g., varied in response to an input from a temperature sensor with the thermal block 2 (not illustrated).

Accordingly, the substrate 12 can be heated by the heating lines 17. Due to thermal communication between the substrate 12 and the sealing foil 7, the sealing foil 7 and wells 5, respectively, can be heated by thermal conduction between the substrate 12 and sealing foil 7. The sealing foil 7 may also be heated by radiation from the area of the substrate 12 and convection of hot air within the wells 5.

The heating element 16 is supported by mount 22 fixedly securing the heating element 16 to the thermal block 2. Stated more particularly, the mount 22 can be used in a clamp design forcing the substrate 12 onto the rim portions 8 of the wells 5 so as to apply a desired clamping force to the multi-well plate 4. The mount 22 may thus exert sufficient pressure to secure the multi-well plate 4 against the upper face 3 of the thermal block 2 with a view of improving thermal communication between the multi-well plate 4 and the thermal block 2. Hence, the clamping force exerted by the mount 22 on the multi-well plate 4 can improve the sealing effect of the sealing foil 7 and additionally provide for a good thermal contact between the multi-well plate 4 and the thermal block 2 to make the heat distribution uniform.

The system 1 may further include a detection arrangement 23 to optically detect emission beams emitted from contents of the wells 5. The emission beams 41 propagate along an emission beam path 40 running through the heating element 16 and the sealing foil 7 of the wells 5. The detection arrangement 23 includes one or more excitation sources (not illustrated) to excite emission of fluorescence light by the reaction products contained in the wells 5 and one or more detectors (not illustrated) to optically detect reaction products such as, but not limited to, a CCD camera. The optically transparent substrate 12 and the optically transparent sealing foil 7 allow excitation light to be transmitted to the reaction products contained in the wells 5 and emitted fluorescent light from the reaction products to be transmitted back to the one or more detectors, e.g., during thermally cycling the samples.

In the heating arrangement 11, the heating lines 17 can be disposed within the emission beam path 40 along which the emission beams 41 propagate to the detection arrangement 23. In that, as seen along the emission beam path 40, at least one well 5 is crossed by at least one heating line 17. In order to essentially avoid interference of the heating lines 17 with the optical detection of the emission beams 41, a width of individual heating lines 17 amounts to a few 10 μm. Additionally, adjacent heating lines 17 and/or adjacent portions of individual heating lines 17 have an inter-distance from some 100 micrometers to some millimeters. The thin heating lines may be, e.g., made of metallic material such as platinum or platinum alloy.

In the heating arrangement 11, the substrate 12 may be, e.g., made of glass such as borosilicate glass and may, e.g., have an optical transmission of more than 80%, preferably more than 85%, when operating the system 1 to optically detect reaction products contained in the wells 5.

Otherwise, a covered portion of an irradiated opening area 43 of the opening 42 of each of the wells 5 covered by one or more of the opaque heating lines 17 is less than 20%, in particular less than 10%. The irradiated opening area 43 is a cross-sectional area of the opening 42 orthogonal to the emission beam 41 emitted by the liquid sample 6 contained in the well 5. In this example, the irradiated opening area 43 is identical to the geometric cross-sectional area of the opening 42. Accordingly, the non-covered portion of the irradiated opening area 43 which can be transmitted (penetrated) by the emission beam 41 is smaller than the irradiated opening area 43 and corresponds to the irradiated opening area 43 reduced by the heating lines 17 covered portion thereof.

With particular reference to FIG. 2, an exemplary embodiment of the heating arrangement 11 of the system 1 of FIG. 1 is explained. Accordingly, the resistive heating element 16 includes a plurality of heating lines 17 formed on the upper face (side) 15 of the substrate 12. The heating lines 17 are narrow lines in parallel arrangement with respect to each other which on their opposing ends are collectively coupled to strip-like busses 24. The busses 24 then are coupled to controller 18 by means of the electric lines 19. It should be understood that the heating lines 17 can have various configurations according to the specific demands of the user. Alternatively, the resistive heating element 16 may be, e.g., patterned as continuous line forming a single circuit path.

Electric contact between the heating lines 17 and busses 24 may be reached by soldering, sticking, bonding, clamping or any other method for connecting electric structures. Particularly, neighboring heating lines 17 can have an inter-distance of some 100 μm to some millimeters. Individual heating lines 17 can have a width of a few 10 μm. The busses 24 can be divided into a plurality of segments connected to individual sets of heating lines 17 so as to selectively contact each set of heating lines 17.

In the system 1, due to their greater exposure to the atmosphere and/or other system components, outer wells 5 typically have a lower temperature than inner wells 5 of the multi-well plate 4. In order to compensate such edge effects, according to another embodiment, the heating lines 17 are operable to yield a non-uniform area density of heating power (heating power per area unit) with respect to the lower face 14 of the substrate 12 to obtain a uniform (homogenous) temperature of the wells 5.

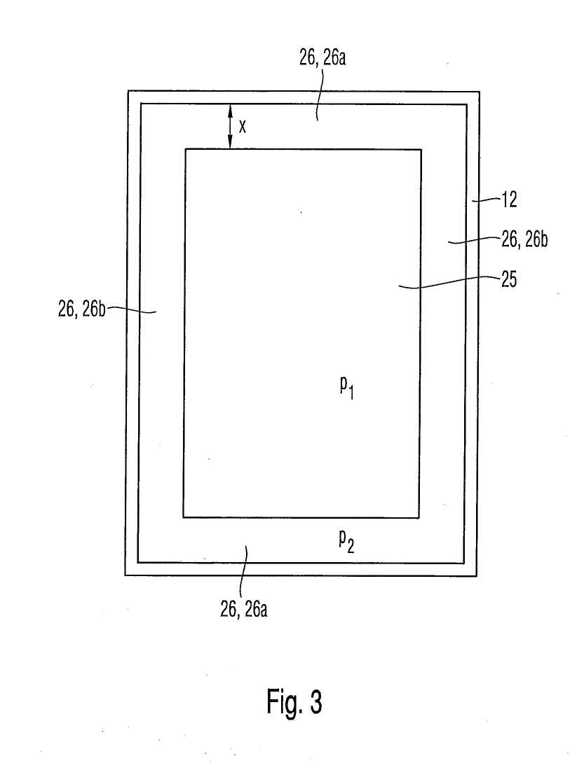

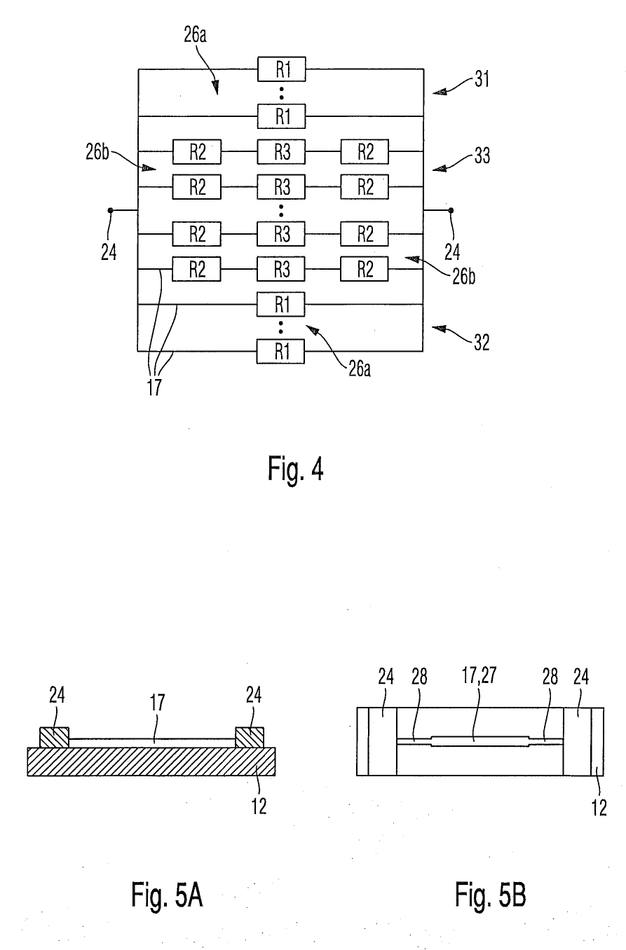

With particular reference to FIG. 3, an exemplary non-uniform area density of heating power of the heating lines 17 with respect to a unit area of the lower face 14 of the substrate 12 to compensate for edge effects is illustrated. Accordingly, in the heating element 16 the heating lines 17 are operable to yield a first area density (p₁) of heating power in a first region of the substrate 12 being lower than a second area density (p₂) of heating power in a second region of the substrate. The first region of the substrate 12 is a central region 25 located above the wells 5 of the multi-well plate 4. The second region of the substrate 12 is an edge region 26 surrounding the central region 25. As detailed in FIG. 4, the edge region 26 includes a first portion 26a corresponding to heating lines 17 having a first electric resistance (R1) and a second portion 26b corresponding to second sections 28 of heating lines 17 having a second electric resistance (R2). Contrary to the first portion 26a of the edge region 26, wells are also located under the second portion 26b of the edge region 26 (in addition to the central region 25).

A ratio of the first area density (p₁) of heating power to the second area density (p₂) of heating power may be, e.g., in a range of from 1 to 1.5 through 1 to 10, in particular in a range of from 1 to 2 through 1 to 3, to thereby obtain a homogenous temperature of the wells 5. The footprint of the substrate 12 effectively used for heating the wells 5 can be about 127 mm in length and about 85 mm in width. A linear dimension (x) of the edge region 26 may be in a range of from 4 to 20 mm and preferably amounts to about 6 mm.

In order to obtain a non-uniform area density of heating power, individual heating lines 17 can be designed to have a varying electric resistance over their extensions.

With particular reference to FIG. 4, depicting an equivalent circuitry, various electric resistances of individual heating lines 17 to yield a different non-uniform area density of heating power as indicated in FIG. 3 are illustrated. Accordingly, the heating lines 17, depending on their specific locations, can have a first electric resistance R1 or, alternatively, can have a first section 27 having a third electric resistance R3 sandwiched in-between two second sections 28 having a second electric resistance R2 with the third electric resistance R3 being higher than the first electric resistance R1 being higher than the second electric resistance R2 (R3>R1>R2). Stated more particularly, each heating line 17 of a first set 31 of heating lines 17 located at the one bus 24-free side of the heating element 16 and a second set 32 of heating lines 17 located at the other bus 24-free side of the heating element 16 has the first electric resistance R1, while each heating line 17 of a third set 33 of heating elements sandwiched in-between the first and second sets 31, 32 has the second and third resistances R2, R3. The first and second sets 31, 32 of heating lines 17 correspond to the first portion 26a of the edge region 26. The third set 33 of heating lines 17 corresponds to the central region 25 and second portion 26b of the edge region 26. Stated more particularly, with respect to the third set 33 of heating lines 17, the first sections 27 of the heating lines 17 correspond to the central region 25 while the second sections 28 of the heating lines 17 correspond to the second portion 26b of the edge region 26.

The varying electric resistance of individual heating lines 17 of the third set 33 of heating lines 17 of the heating element 16 of FIG. 2 can be obtained by varying specific line characteristics such as line width, line height and/or line material as is further detailed below.

Reference is made to FIG. 5A and FIG. 5B which are schematic diagrams depicting a cross-sectional view (FIG. 5A) of a variant of the heating element 16 and a top view thereof (FIG. 5B) to illustrate a varying line width of individual heating lines 17 of the heating element 16 of the third set 33 of heating lines 17 of FIG. 2. The line width is a linear dimension perpendicular to the direction of the heating line 17 in a plane of the substrate 12 as, e.g., defined by the upper face 15 thereof. Accordingly, each heating line 17 of the third set 33 comprises the first (inner) section 27 located in-between the second (outer) sections 28 wherein the first section 27 has a bigger line width than the second sections 28 yielding a higher electric resistance per length in the second sections 28 than in the first section 27.

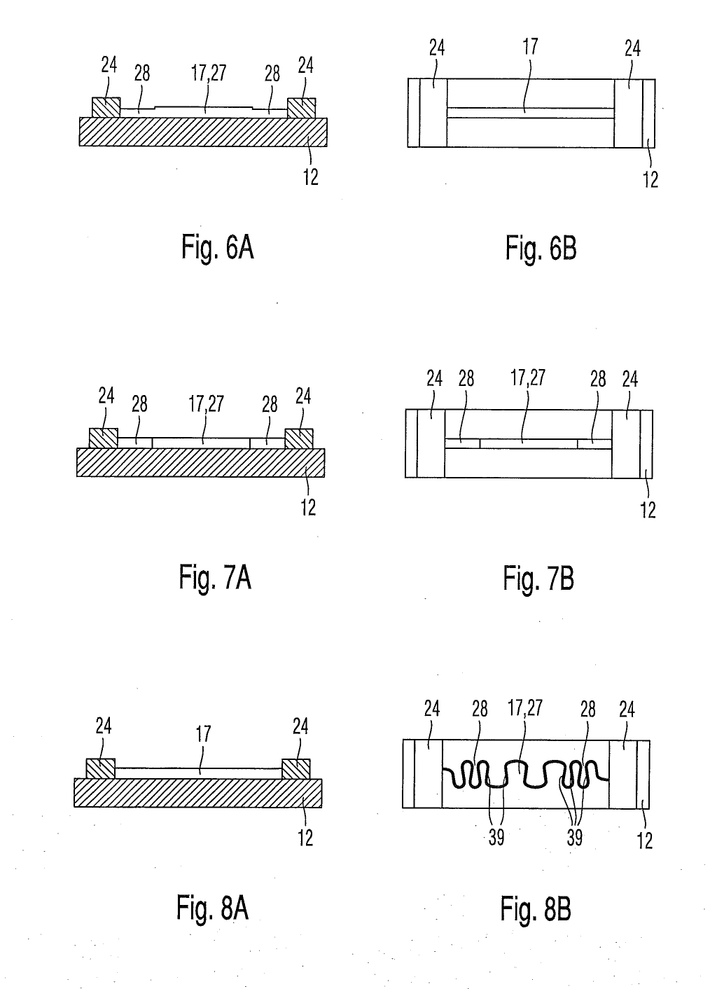

Reference is made to FIG. 6A and FIG. 6B which are schematic diagrams depicting a cross-sectional view (FIG. 6A) of another variant of the heating arrangement 11 and a top view thereof (FIG. 6B) to illustrate a varying line height over the extension of an individual heating line 17 of the heating element 16 of the third set 33 of heating lines 17 of FIG. 2. The line height is a linear dimension perpendicular to the direction of the heating line 17 and orthogonal to the plane of the substrate 12 as, e.g., defined by the upper face 15 thereof. Accordingly, each heating line 17 of the third set 33 comprises the first (inner) section 27 located in-between the second (outer) sections 28 wherein the first section 27 has a bigger line height than the second section 28 yielding a higher electric resistance per length in the second sections 28 than in the first section 27.

Reference is made to FIG. 7A and FIG. 7B, which are schematic diagrams depicting a cross-sectional view (FIG. 7A) of another variant of the heating arrangement 11 and a top view thereof (FIG. 7B) to illustrate a varying line material of the extension of an individual heating line 17 of the heating element 16 of the third set 33 of heating lines 17 of FIG. 2. Accordingly, each heating line 17 of the third set 33 comprises the first (inner) section 27 located in-between the second (outer) sections 28 in which the first section 27 is made of a first material while the second sections 28 are made of a second material wherein the second material has a higher electric resistance per length than the first material.

In order to obtain a varying electric resistance of individual heating lines 17, each of the heating lines 17 may include first and second sections 27, 28 having a varying width and/or a varying height and/or a varying material over its extension. The first sections 27 of the heating lines 17 of the third set 33 of heating lines 17 are associated to the central region 25 of the substrate 12 while the first and second sets 31, 32 of heating lines 17 are associated to the first portion 26a of the edge region 26 and the second sections 28 of the third set 33 of heating lines 17 are associated to the second portion 26b of the edge region 26.

In order to obtain a non-uniform area density of heating power, a plurality of heating lines 17 and/or plural portions of individual heating lines 17 can be designed in such a manner so as to have a varying area density of the heating lines 17 and/or a varying area density of plural portions of individual heating lines 17. As used herein, the term “area density” refers to a density of the heating lines 17 and/or portions thereof with respect to an area of the substrate 12 as, e.g., defined by the upper face 15 thereof.

Reference is made to FIG. 8A and FIG. 8B, which are schematic diagrams depicting a cross-sectional view (FIG. 8A) of the heating arrangement 11 and a top view thereof (FIG. 8B) to illustrate a varying area density of plural portions 39 of an individual meandering heating line 17 of the heating element 16 of the third set 33 of heating lines 17 of FIG. 2. Accordingly, each heating line 17 of the third set 33 comprises a meandering first (inner) section 27 located in-between meandering second (outer) sections 28. In that, the first section 27 has neighboring portions 39 which have a bigger inter-distance corresponding to a smaller area density of the portions 39 of the heating line 17 than neighboring portions 39 of each of the second sections 28. Accordingly, compared to heating power of the first section 27, a higher heating power per area unit can be obtained in the second sections 28.

In order to obtain a non-uniform area density of heating power, a varying area density of plural heating lines 17 and/or a varying area density of plural portions of individual meandering heating lines 17 can be combined with a varying electric resistance of individual heating lines 17 as illustrated in combination with FIGS. 5A-B, 6A-B and 7A-7B.

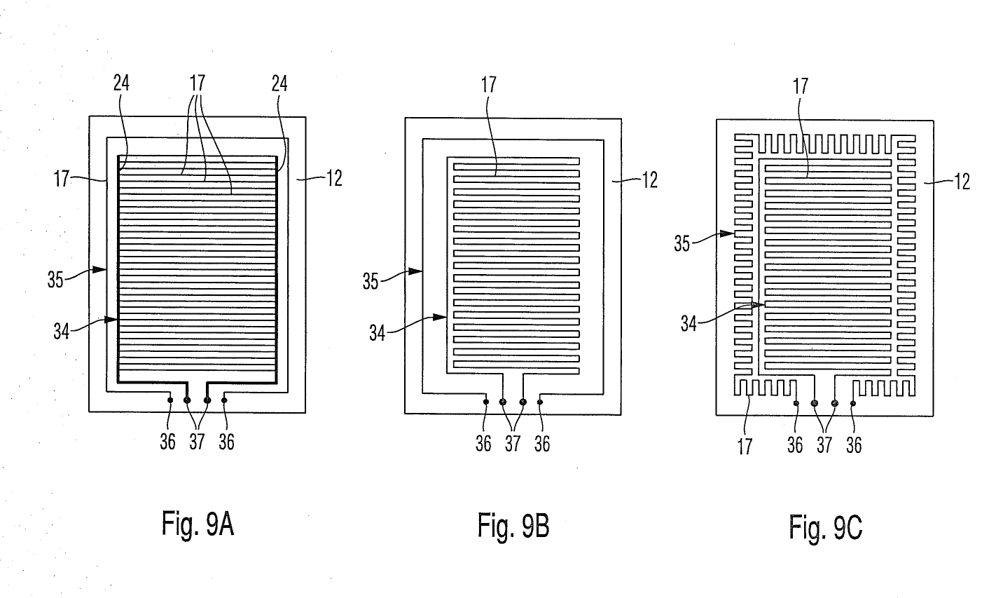

Reference is made to FIG. 9A through 9C, which are schematic diagrams depicting top views of various variants of the heating element 16 of FIG. 2. Accordingly, the heating element 16 includes two separate (independent) heating circuits 34, 35, each of which having separate electric connectors 37, 36 which are selectively connectable to one or more electric power sources. The (first) inner heating circuit 34 is being adapted to heat the central region 25 of the substrate 12 while the (second) outer heating circuit 35 is being adapted to heat the edge region 26 thereof.

Specifically, with particular reference to FIG. 9A, in one variant, the inner heating circuit 34 includes a plurality of heating lines 17 which are narrow lines in parallel arrangement with respect to each other which on their opposing ends are collectively coupled to busses 24 ending in two inner connectors 37. Otherwise, the outer heating circuit 35 is a continuous heating line 17 forming a single circuit path ending in two outer connectors 36.

Specifically, with particular reference to FIG. 9B, in another variant, the inner heating circuit 34 is a continuous meandering heating line 17 forming a single circuit path ending in two inner connectors 37. Otherwise, the outer heating circuit 35 is a continuous non-meandering heating line 17 forming a single circuit path ending in two outer connectors 36.

Specifically, with particular reference to FIG. 9C, in yet another variant, the inner heating circuit 34 is a continuous meandering heating line 17 forming a single circuit path ending in two inner connectors 37. Otherwise, the outer heating circuit 35 is a continuous meandering heating line 17 forming a single circuit path ending in two outer connectors 36.

The inner and outer heating circuits 34, 35 can be selectively operated in parallel or consecutively according to the specific demands of the user in order to yield a higher area density of heating power in the edge region 26 than in the central region 25 of the substrate 12. Each of the separate heating circuits 34, 35 can, e.g., have a varying area density of plural heating lines 17 and/or a varying area density of plural portions of individual meandering heating lines 17 as illustrated in combination with FIGS. 8A-8B and/or can be combined with a varying electric resistance of individual heating lines 17 as illustrated in combination with FIGS. 5A-B, 6A-B and 7A-7B.

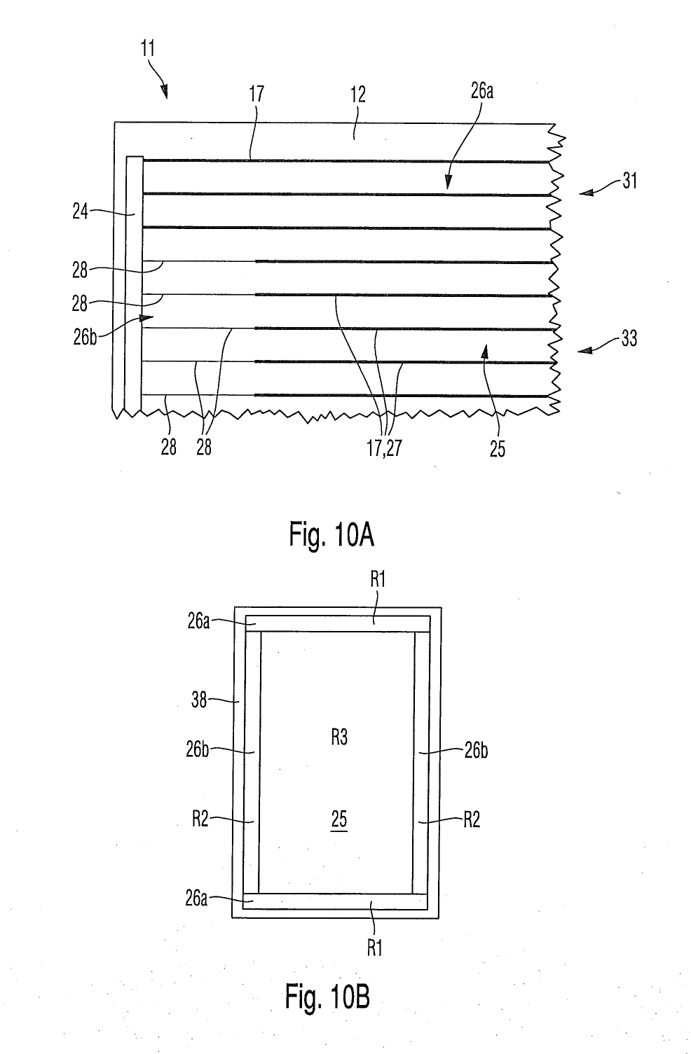

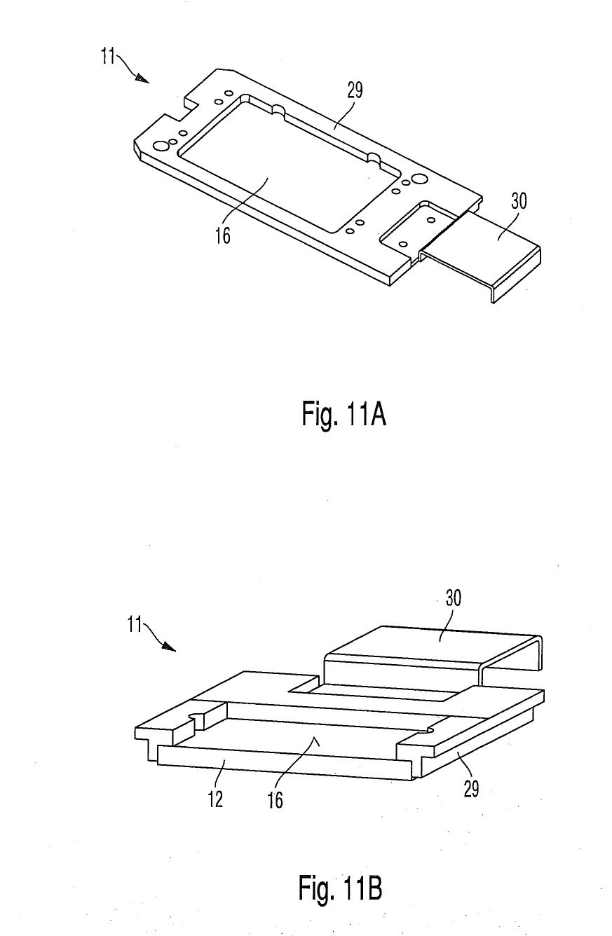

Reference is made to FIG. 10A and FIG. 10B, which are schematic diagrams illustrating a specific embodiment of the heating element 16 of FIG. 1 corresponding to the embodiment as illustrated in FIGS. 4 and 5A-5B. With particular reference to FIG. 10A, the heating element 16 includes substrate 12 provided with resistive heating lines 17 to heat the substrate 12 by generating Ohmic heat. The resistive heating lines 17 are narrow lines in parallel arrangement with respect to each other which on their opposing ends are collectively coupled to strip-like busses 24.

In FIG. 10A, the first and third sets 31, 33 of heating lines are shown for the purpose of illustration only. While not shown in FIG. 10A, the second set of heating lines 17 is similar to the first set 31 of heating lines 17. The first set 31 includes a plurality of three heating lines 17 having a similar line width to yield the first electric resistance (R1). While a number of three heating lines 17 are illustrated, the skilled persons will appreciate that the first set 31 may include any other number of heating lines 17 according to the specific demands of the user. Otherwise, each heating line 17 of the third set 33 comprises a first section 27 located in-between second sections 28 wherein the first section 27 has a bigger line width than the second sections 28 yielding a higher electric resistance per length in the second sections 28 than in the first section 27. The heating lines 17 are formed by use of thin layer and lithographic technology. The heating lines 17 are made of platinum (Pt) and have a height of about 0.3 μm. The busses 24 are, e.g., made of Gold (Au) having a thickness of about 2 μm and a width of about 2 mm. The heating lines 17 can, e.g., be electrically connected and wired via the busses 24 by means of soft solder connections (not illustrated).

The heating lines 17 of the first set 31 have a width of about 119 μm. The first section 27 of each of the heating lines 17 of the third set 33 has a width of about 68 μm, while the second sections 28 thereof have a width of about 28 μm. The various widths of individual heating lines 17 are designed in such a manner so as to have a ratio of area density of heating power of the central region 25 to the edge region 26 of 1:2.5 to reach homogeneity of the temperature of the wells 5. An inter-distance between adjacent heating lines 17 is about 1.125 mm. An optical transmission of the heating arrangement 11 of FIG. 10 is determined by the transmission of the substrate 12 and shadowing effects of the heating lines 17. All in all, the central region 25 has an optical transmission of about 86% while the edge (region) area or second portion 26b has an optical transmission of about 89%. The wells 5 of the multi-well plate 4 are positioned below the central region 25 and the second portion 26b of the edge region 26, i.e. below the third set 33 of heating lines 17 including the first sections 27 and the second sections 28. Otherwise, no wells 5 are located below the first portion 26a of the edge region 26, i.e. below the first set 31 (and second set) of heating lines 17.

FIG. 10B illustrates the overall dimensions of the heating element 16 including the various heating areas as defined by the different electric resistances R1, R2, and R3 with an area. Accordingly, the substrate 12 is, e.g., made of borosilicate glass having a rectangular size of, e.g., 125×86 mm and a thickness of, e.g., 6 mm. Specifically, the central region 25 having the heating lines (filaments) with the third resistance R3 has a rectangular size of, e.g., 107×65 mm, while the second portion 26b of the edge region 26 having the heating lines (filaments) with the second resistance R2 has a rectangular size of, e.g., 107×6 mm and the first portion 26a of the edge region 26 having the heating lines (filaments) with the first resistance R1 has a rectangular size of, e.g., 77×6 mm. The substrate 12 further includes a border 38 surrounding the edge region 26 not provided with heating lines 17. Those of skill in the art will appreciate that the heating arrangement 11 and heating lines 17 can be formed in dimensions other than those specified herein.

Reference is made to FIGS. 11A-11B, which are a perspective view (FIG. 11B) and a cross-sectional view of the heating arrangement 11 of FIGS. 10A-10B. Accordingly, the heating arrangement 11 includes a frame 29 surrounding the optically transparent substrate 12 made of thermally low-conductive material such as, but not limited to, plastic material. The frame 29 is provided with a handle 30 to, e.g., manually or robotically place the heating arrangement 11 on the multi-well plate 4. The heating arrangement 11 may be embodied as a (e.g. modular) system component which can be readily used for multi-well plates 4 that are similar or different in array sizes.

In the following a specific example of the optical transmission of the heating element 16 as illustrated in FIGS. 10A-10B is given. In this example, it is assumed that the substrate 12 is made of borosilicate glass. It is further assumed that a diameter of each of the wells 5 at their opening is 1.2 mm which is fully irradiated by radiation emitted from samples contained in the wells 5. It is yet further assumed that, relative to the emission beam path 40, each well 5 is crossed by one heating line 17. The optical transmission of the heating element 16 thus is influenced by the substrate 12 and by the heating lines 17 shadowing and/or scattering light. Furthermore, since no wells 5 are located below the first and second sets 31, 32 of heating lines 17, in the following, the term “edge region 26” refers only to that part of the heating element 16 provided the second sections 28 of the third set 33 of heating lines 17 corresponding to the second portion 26b of the edge region 26. Otherwise, the term “central region 25” refers to that part of the heating element 16 provided with the first sections 27 of the third set 33 of heating lines 17.

Accordingly, an optical transmission (OT_{sub}) of the substrate 12 made of borosilicate glass amounts to 92% as measured with a conventional spectrometer.

For example, in a multi-well plate 4 provided with 1536 wells 5, shadowing with respect to the opening area of one well 5 caused by one heating line 17 can be obtained by calculating a ratio given by an area A₁ where one heating line 17 (partially) covers the opening area of one well 5 and the opening area A₂ of one well 5. Being different for the central region 25 and the edge region 26, it follows:

Central region 25: Error! Objects cannot be created from editing field codes.

Edge region 26: Error! Objects cannot be created from editing field codes.

Accordingly, a heating line-covered portion of the opening area 43 of an individual well 5 amounts to 7.2% in the central region 25 and to 3.0% in the edge region 26.

As a result, an optical transmission OTₕₗ of the heating element based on the heating lines 17 can be obtained. Being different for the central region 25 and the edge region 26, it follows:

Central region 25: OTₕₗ=92.8%

Edge region 26: OTₕ₁=97.0%

As a result, a total optical transmission OTₜₒₜ of the heating element 16 can be obtained. Being different for the central region 25 and the edge region 26, it follows:

Central region 25: OTₜₒₜ=0.92·0.928=85.4%

Edge region 26: OTₜₒₜ=0.92·0.970=89.2%

Hence, the (theoretical) total optical transmission of the heating element 16 amounts to 85.4% in the central region 25 and to 89.2% in the edge region 26.

The total optical transmission OTₜₒₜ of the heating element 16 can be measured by the following procedure:

First, a predetermined amount of a fluorescent solution (fluorophore) such as fluorescein is filled into the wells 5 of the multi-well plate 4. Then, emission of fluorescence light is excited, followed by detecting the fluorescence light of each of the wells without heating element 16 and with heating element 16 positioned between the wells 5 and the detection arrangement 23.

A total optical transmission OTₜₒₜ of the heating element 16 related to the specific geometric conditions of the wells 5 can then be obtained by calculating a square root of the quotient of intensities of fluorescence light with heating element 16 (F₁) and without heating element 16 (F₂): Error! Objects cannot be created from editing field codes.

Another method to measure the optical transmission of the heating element 16 uses an absorption spectrometer or absorption photometer. To measure the total transmission, the heating element is positioned into the beam of the spectrometer or photometer with the area of interest (center region (area) 25 and maybe edge area or second portion 26b) located in the center of the beam. The provided beam size must be large enough to average multiple heating lines. The transmission of the heating element is calculated by the difference of the absorption with heating element and without heating element in the beam (air reference).

With continued reference to this example, various variations of the total optical transmission OTₜₒₜ can be obtained:
1) Variation between thicker and thinner heating lines 17: 85.4%−89.2%=3.8%
2) Variation between thicker heating lines 17 and regions without heating lines 17: 92.0%−85.4%=6.6%
3) Variation between thinner heating lines 17 and regions without heating lines 17: 92.0%−89.2%=2.8%

The system 1 can readily be calibrated in positioning the heating element 16, exciting fluorescence light and detecting of fluorescence light with and without filling a predetermined amount of a standard fluorescent solution into the wells 5 of the multi-well plate 4 so as to determine background signals. Such calibration procedure can, e.g., be repeated several times, e.g., to determine whether the optical transmission is influenced by tolerances in positioning the heating element 16.

Accordingly, in the system 1 as-above detailed, a plurality of samples 6, e.g., including biological material can be cycled through a pre-defined temperature profile under control of the controller 18 to accomplish a polymerase chain reaction of nucleic acids contained therein. Specifically, the samples together with specific reagents for amplifying the nucleic acids are filled into the wells 5 of the multi-well plate 4 covered with sealing foil 7. The heating element 16 is placed above the multi-well plate 4 in thermal communication with the sealing foil 7. When cycling the samples through the temperature excursions by use of the thermal block 2, the heating element 16 is heated so as to have a temperature similar to the thermal block 2. Accordingly, the multi-well plate 4 is heated from both the thermal block and the heating element 16, wherein the heating element 16 is operated to yield a non-uniform area density of heating power with respect to the substrate 12 to compensate for edge effects and to obtain a uniform temperature of the plurality of wells 5. The contents of the wells 5 can be optically detected through the heating element 16. Specifically, optical transmission of the substrate 12 and shadowing and/or scattering effects of the heating lines 17 enable optical detection of the emission beams in a reliable and satisfactory manner, e.g., based on excitation of the sample without relevant interference between heating lines 17 and emission beams 41. The system 1 thus allows the emission beams 41 to be readily detected even in case the heating lines 17 are located within the emission beam path 40. The heating element 16 can thus be easily arranged above the wells 5 without a need to exactly position it in order to keep the heating lines 17 out of the emission beam path 40 and eventually excitation beam path which facilitates positioning of the heating element 16 above the multi-well plate 4. The heating element 16 can readily be used for various multi-well plates 4 having array sizes which are different with respect to each other. The heating arrangement 11 permits the user to optically detect the emission beams 41, e.g., during the course of the reaction without experiencing condensation on the sealing foil 7. It especially permits homogeneous heating of the wells 5 to avoid temperature variations so as to enable similar chemical reactions in the wells 5 to take place.

A major advantage is given by the fact that, due to many small heating lines 17 per length unit instead of only few thicker heating lines, there are only rather small local variations between the total optical transmissions of the heating element 16 from one well 5 to another well 5 thus enabling a highly reliable detection of the contents of the wells 5. Accordingly, the system 1 is much less sensitive to tolerances when positioning the heating element 16. Such effect can be further improved in reducing the thickness of the heating lines 17 as much as reasonably possible, however, limited by self-destruction of the heating lines 17 and/or production limits.

Furthermore, due to the possibility of applying non-uniform heating power, edge effects can advantageously be avoided.

Thus, by the above disclosed embodiments, a system and method for cycling liquid samples through a series of temperature excursions are disclosed. One skilled in the art will appreciate that the teachings can be practiced with embodiments other than those disclosed. The disclosed embodiments are presented for purposes of illustration and not limitation, and the invention is only limited by the claims that follow.

What is claimed is:

1. A system for cycling liquid samples through a series of temperature excursions, comprising:
a plurality of open-top reaction vessels for containing said samples, said reaction vessels being enclosed by one or more covers;
a temperature-controlled thermal block for generating or adsorbing heat thermally coupled to said reaction vessels;
a detection arrangement for detecting radiation disposed in an emission beam path to detect emission beams emitted from said samples received through said one or more covers;
a heating arrangement for generating heat including a heating element disposed between said reaction vessels and said detection arrangement and being thermally coupled to said one or more covers, said heating element including an optically transparent substrate provided with one or more opaque heating lines, said heating lines being disposed in said emission beam path in a manner to obtain a predetermined minimum optical transmission of said heating element; and
a controller, set up to control cycling of the samples.

2. The system according to claim 1, in which said heating element has a minimum optical transmission percentage selected from 50%, 70%, and 85% with respect to said emission beams emitted from said samples.

3. The system according to claim 1, in which a covered portion with respect to an irradiated opening area of individual reaction vessels covered by said one or more heating lines is less than 20%.

4. The system according to claim 1, in which a covered portion with respect to an irradiated opening area of individual reaction vessels covered by said one or more heating lines is less than 10%.

5. The system according to claim 1, in which individual heating lines have a width of less than 150 μm.

6. The system according to claim 1, in which individual heating lines have a width of less than 120 μm.

7. The system according to claim 1, in which individual heating lines have a width in a range of from about 10 μm to about 70 μm.

8. The system according to claim 1, in which at least one of adjacent heating lines and adjacent portions of individual heating lines have an inter-distance of more than 100 μm.

9. The system according to claim 1, wherein said one or more heating lines being operable to yield a non-uniform area density of heating power with respect to an area of said substrate being thermally coupled to said one or more covers.

10. The system according to claim 9, wherein said one or more heating lines have a varying electric resistance over their extensions.

11. The system according to claim 10, wherein said one or more heating lines vary in one or more of the following characteristics selected from the group consisting of line width, line height and line material over their extensions.

12. The system according to claim 9, wherein an area density of at least one of said one or more heating lines and portions of individual heating lines varies with respect to said area of said substrate being thermally coupled to said covers.

13. The system according to claim 12, wherein said heating arrangement includes one or more meandering heating lines.

14. The system according to claim. 9, wherein said one or more heating lines being operable to yield a first area density of heating power in a central region of said substrate being lower than a second area density of heating power in an edge region of said substrate surrounding said central region.

15. The system according to claim 9, wherein said heating arrangement includes at least two heating circuits having separate connectors connectable to one or more power sources.

16. A heating arrangement for heating one or more covers enclosing a plurality of reaction vessels for containing liquid samples, said heating arrangement including a heating element disposed between said one or more covers and a detection arrangement disposed along an emission beam path for detecting emission beams emitted from said samples received through said one or more covers, said heating element including an optically transparent substrate provided with one or more opaque heating lines disposed in said emission beam path in a manner to obtain a predetermined minimum optical transmission of said heating arrangement.

17. A method for cycling liquid samples through a series of temperature excursions, comprising:
providing said liquid samples in a plurality of open-top reaction vessels enclosed by one or more covers;
thermally cycling said samples;
detecting emission beams emitted from said samples and received through said one or more covers along an emission beam path; and
heating said one or more covers by a heating arrangement including a heating element having a transparent substrate provided with one or more opaque heating lines disposed in said emission beam path in a manner to obtain a predetermined minimum optical transmission of said heating element.

18. The method of claim 17, further comprising operating said one or more heating lines to yield a first area density of heating power in a central region of said substrate being lower than a second area density of heating power in an edge region of said substrate surrounding said central region.
